(19) United States
(12) Patent Application Publication
(10) Pub. No.: US 2020/0397266 A1
(43) Pub. Date: Dec. 24, 2020

(54) APPARATUS AND METHOD FOR ENHANCED TISSUE VISUALIZATION

(71) Applicant: TransEnterix Surgical, Inc., Morrisville, NC (US)

(72) Inventor: Kevin Andrew Hufford, Cary, NC (US)

(21) Appl. No.: 15/917,896

(22) Filed: Mar. 12, 2018

Related U.S. Application Data
(60) Provisional application No. 62/470,110, filed on Mar. 10, 2017.

(51) Int. Cl. A61B 1/05; A61B 1/00; A61B 1/07; A61B 1/06; A61B 17/34

(52) U.S. Cl. CPC A61B 1/05; A61B 1/00045; A61B 1/00039; A61B 17/34; A61B 1/0684; A61B 1/0638; A61B 1/0646; A61B 1/07

(57) ABSTRACT

A medical imaging system includes an image sensor, a first source positioned to direct light in a first wavelength range onto a work site, and a second source positioned to direct light in a second wavelength range onto a work site. The image sensor is configured to capture light emitted or reflected from the work site during illumination of the first source and during illumination of the second source. As a result, the user can view both broad spectrum images of the illuminated work site and narrow spectrum (e.g. fluorescent) images of the work site captured using a single image sensor on the system's camera display.

This application claims the benefit of U.S. Provisional Application No. 62/470,110, filed Mar. 10, 2017.

### FIELD OF THE INVENTION

The invention relates generally to the field of medical imagers such as endoscopes and laparoscopes. More specifically, the invention relates to the use of medical imagers for hyperspectral imaging for use in tissue identification, diagnosis, and other procedures.

### BACKGROUND

During surgical procedures, including laparoscopic surgical procedures using manual or robotic surgical techniques, an endoscopic/laparoscopic camera is positioned within the body cavity to capture images of the operative site. Camera systems used for endoscopic/laparoscopic imaging (referred to here as “endoscopic imaging systems” or “medical imaging systems”) typically include a light source that projects light onto body tissue and one or more image sensors that receive light reflected from the body tissue. The image sensor generates image signals corresponding to images of the operative site, and the images are displayed on a video monitor observed by the surgeon during the course of the procedure.

The image sensor includes an array of photosensors. A color filter array (“CFA”) may be positioned over the array of photosensors, allowing each photosensor to detect only the color that can pass through the portion of the filter that is covering it. The CFA for a medical imager is commonly a standard Bayer filter, in which an array of red, green and blue color filters are arranged over the grid of photosensors comprising the imager. The filters of the array are arranged in the red-green-blue-green, or “RGBG” pattern illustrated in FIG. 1A, where filters marked R, G and B are transmissive to red, or green or blue light exclusively, exposing each pixel of the photosensor array to only one of the three color bands. The medical imaging system interpolates the full color image from the sensed array using multiple algorithms.

Fluorescence is the emission of light by a substance when it is exposed to photons of another wavelength. Typically fluorescent molecules absorb electromagnetic radiation at one wavelength and emit it at another, longer, wavelength. Fluorescence imaging using a fluorescent dye is sometimes used in medical imaging to allow the surgeon to see particular tissue types or structures within the operative field. For example, indocyanine green (ICG) and methylene blue (MB) are fluorescent substances that will emit fluorescence when exposed to near-infrared right. The tissues/structures to be identified are marked or infused using the fluorescent substance, and the area is exposed to light in a band of the near infrared range. The marked/infused tissue absorbs and then emits fluorescence, which may be detected by an image sensor.

Another type of fluorescence, called auto-fluorescence, relies on the native fluorescence properties of certain tissues rather than added fluorescent markers.

The wavelength of light needed for fluorescence depends on the fluorescent material or agents. Some materials fluoresce when exposed to near infrared light, others fluoresce when exposed to UV light, while others fluoresce when exposed to light of other wavelengths.

This application describes improved methods and devices allowing use of a medical imager to gather multi-spectral information, which may be outside the visible light range, for use in tissue identification, diagnosis, and other purposes.

### BRIEF DESCRIPTION OF THE DRAWINGS

FIG. 1(a) shows a pixel pattern for a standard Bayer color filter array;

FIG. 1(b) shows a pixel pattern for an RGB-IR imager;

FIGS. 2(a), 2(b) and 2(c) are exemplary timing diagrams showing synchronization of image capture with multiple wavelengths of light (which may be from tunable light sources and/or from multiple light sources).

FIG. 3(a) schematically shows an exemplary scope system.

FIG. 3(b) schematically shows an exemplary scope head.

FIG. 4 shows features of an imaging system incorporating aspects of the disclosed invention.

FIG. 5 schematically illustrates a trocar with illumination features extending through an incision in a patient, and a scope extending through the trocar.

### DETAILED DESCRIPTION

General features of a multi-spectral imaging system incorporating features described in this application may be understood with reference to FIG. 4. FIG. 4 shows an endoscopic imaging system 10 having a scope 12 with an elongate shaft positionable within a body cavity of a patient, an image sensor, a light source for generating light that impinges on tissue at the operative site, and a display 16 that displays images captured by the image sensor. A camera control unit 18 is coupled via a cable 20 to the camera head of the scope 12.

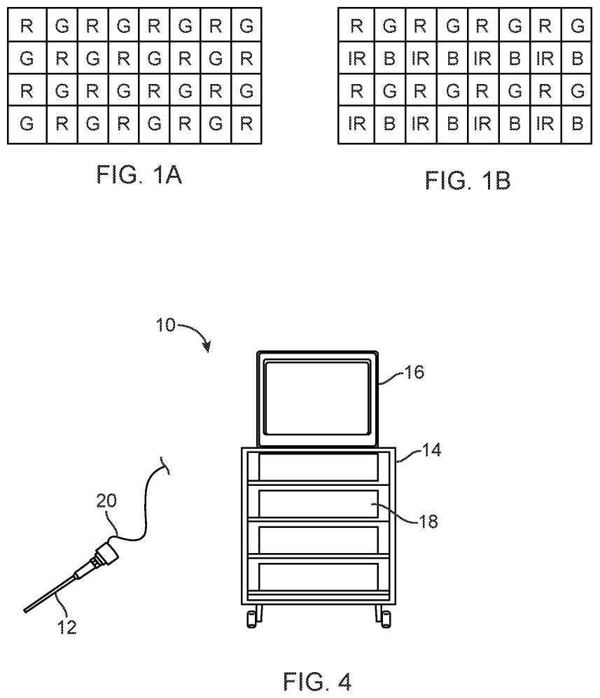

The multi-spectral imaging system includes an image sensor such as a CMOS or CCD imaging chip disposed on/in the camera head of the scope 12. The scope may be of the “distal sensor” or “chip on tip” variety, in which case the sensor is positioned at the distal end of the scope. Alternatively, the sensor may be proximally positioned and arranged such that optical fibers carry reflected light from the tissue to the sensor. A multi-spectral filter array is positioned to filter light reflected onto the image sensor so that different pixels of the image sensor receive light of different wavelengths. In an exemplary embodiment, the filter array has the RGB-IR pattern shown in FIG. 1B (the OmniVision OV4682), allowing red, green, blue and infrared light to be received by the pixels underlying the red, green, blue and infrared portions of the filter, respectively. In alternative embodiments, certain filters in the filter mosaic are arranged to place narrower band filters over certain pixels (e.g. those that allow passage only of the reflected wavelength of ICG fluorescence to pass) while still providing broad spectrum imaging with adjacent pixels. Some embodiments might include filter mosaics having filter patterns that are different in different areas of the imager to provide different wavelengths of light at different locations or regions of interest within the body. In some embodiments, the scope might include multiple image sensors and use at least one beam splitter, shutter, or movable mirror to direct captured light to a select one of the image sensors.

The term “light” as used herein means electromagnetic radiation in the visible, near-visible, infrared or ultraviolent range of the electromagnetic spectrum. The ultraviolet spectrum includes wavelengths from about 100-400 nm. The visible spectrum includes wavelengths in the range of about 400 nm to about 700 nm. The infrared spectrum includes wavelengths in the range of about 700 nm to 3000 nm. For applications described herein, infrared radiation is most suitable in the range of 700 nm to 1400 nm, and more preferably the range of 700 nm to 1000 nm.

The light source may be any type of source that is suitable for the wavelengths of light desired to be captured using the sensor. Examples include, broad-spectrum sources (i.e. sources emitting light with wavelengths spanning a broad range of the EM spectrum), narrow spectrum sources (i.e. sources emitting light with wavelengths spanning a narrow range of the EM spectrum), visible sources (RGB), ultraviolet, infrared etc. The form(s) of the light source(s) may be, but are not limited to, LED, fiber optic light source, lasers, laser diodes, and tunable lasers.

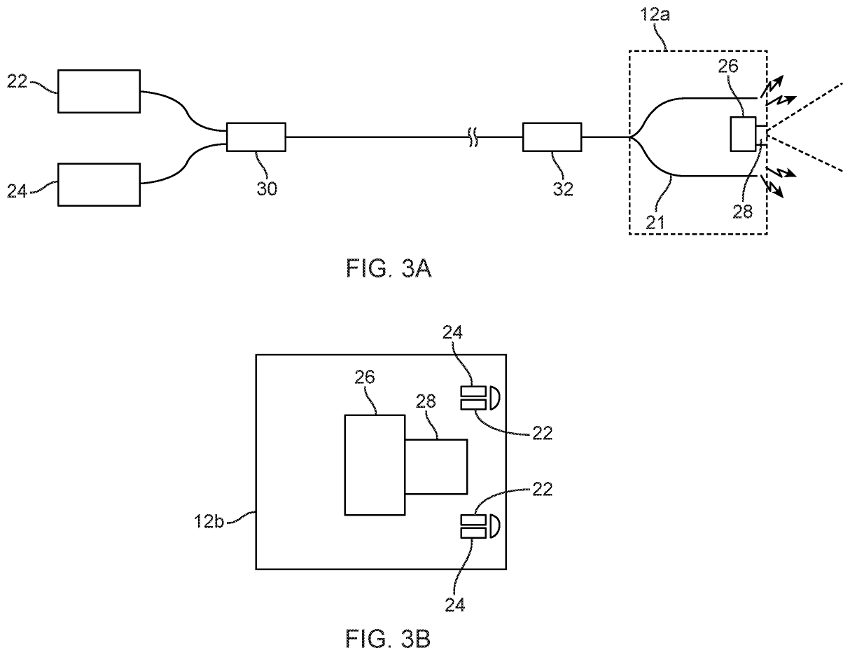

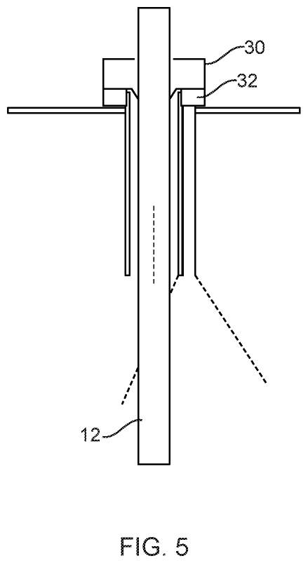

In preferred embodiments of an imaging system 10, combinations of such sources will be used in a single system, with a first source emitting light having wavelengths in a first band and a second source emitting light having wavelengths in a second band. In some embodiments, the first and second bands are not overlapping bands, whereas in other embodiments the bands may have some wavelengths in common. Combinations of light sources might include one in the visible range and one in the infrared range FIGS. 3(a) and 3(b), which show a system that uses white LED's as the source for light in the visible range, and 760 nm LED's (infrared range) as a second source, thus providing first and second sources for emitting light which may have non-overlapping wavelengths. Other configurations might use a UV light source in combination with the visible source or the infrared source, while still other configurations will use three light sources, such as one light source in the visible range, one in the infrared range, and one in the UV range.

In systems using two or more light sources, the system may be configured such that lighting of the work site by given sources is time synchronized and alternated in an appropriate pattern. For example, light from a broad spectrum source might be caused to impinge on the work site for a period of time to allow standard-type imaging of the operative site, with the standard 2D or 3D image shown on the display 16, and then the site is exposed to light from a narrow spectrum source selected to allow certain tissues, structures, etc. to be seen on the display 16. The narrow spectrum source might be an IR or UV source that emits light within a narrow band of the EM spectrum selected to match/encompass the wavelength of light needed to cause fluorescent emissions of the target material.

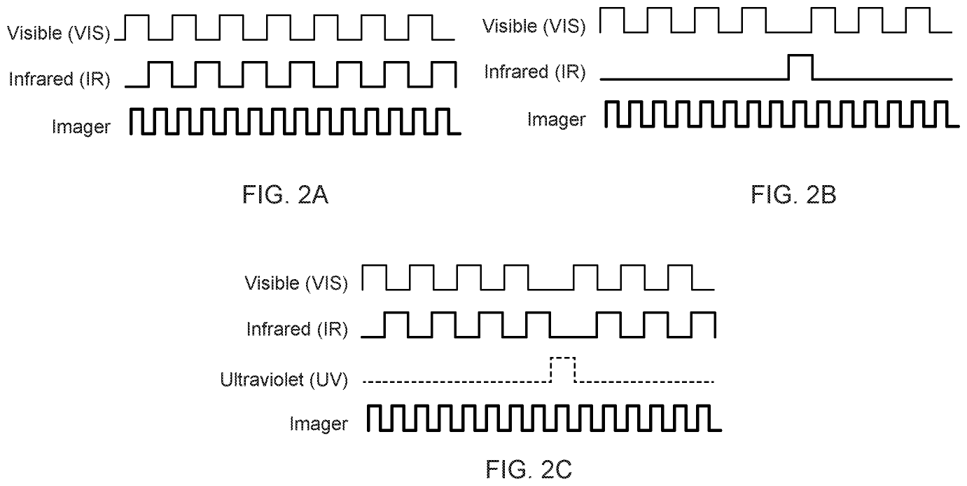

As one example, a broad spectrum source might be caused to impinge on the work site for one frame, and a narrow spectrum source caused to impinge on the work site for the following frame, with the pattern then repeating. See FIG. 2(a). An alternative illumination pattern might call for broad spectrum illumination for a series of frames, narrow spectrum light (shown as IR in the drawing) for a frame as shown in FIG. 2(b), with broad spectrum illumination repeating for a series of frames and the pattern then repeating. In this example, intermittent flashes of the narrow band light allow the user to obtain periodic updates on subsurface anatomy or other anatomy that can be best visualized using fluorescence during the course of a procedure. A third example shown in FIG. 2(c) is similar to the FIG. 2(a) example, but adds periodic flashes of a different narrow band of light (here shown as UV light) to allow visualization of tissues/substances that fluoresce when exposed to light in that range of the EM spectrum. Typically the IR and UV exposures in these examples are within a narrow band of the EM spectrum selected to match/encompass the wavelength of light needed to cause fluorescent emissions of the target material. In each of these examples, the camera continues to collect image data throughout the course of the alternating light exposures. Note that in the listed examples, the broad spectrum source might be replaced with a narrow spectrum source producing wavelengths in the visible range and not overlapping with the wavelengths produced by the second or third light sources.

In other configurations or operating modes, the user selects between a first mode in which the work site is exposed to light from the first source, and a second mode in which the work site is exposed to light from the second source.

The system 10 includes a switching mechanism and associated electronics to carry out switching between illumination of the operative site using a first light source and illumination using a second (or, where applicable, third) one of the light sources in accordance with the programmed illumination cycles or in response to user input. The switching mechanism may utilize a controller that alternates between the light sources by directly controlling the sources (turning them on and off), or it may utilize a controller to control one or more shutters to block the light paths of the light sources, or it might employ one or more digital micromirror devices (DMD). The use of one or more DMDs may allow for varying the light pattern or to blend the light across the field.

Different configurations and positions may be used for the light sources (which, in the case of fluorescence, are excitation sources for the fluorescent tissue/media). Each light source may be positioned to itself emit the light towards the tissue, or it may be used in combination with optical fibers that carry light from the light source to another location from which the light is emitted towards the tissue. The scope itself may be designed to emit the light from a location on the scope, such as its distal end, in any one of a variety of arrangements. In one example of this, shown in FIG. 3(a) a scope 12 may have optical fibers 21 at or around its distal end (in a circumferential arrangement or otherwise) that carry light from one or more proximally located light source, such as a white LED 22 for visible illumination, and a narrow wavelength LED, laser or tunable laser source 24, which in this embodiment is a 760 nm LED as an infrared source 24. Here the fibers are shown emitting light from the scope head 12a, which also houses the 2D or 3D image sensor 26 and a corresponding lens assembly 28. In modified versions of this embodiment, a third, narrow band, source such as one in a wavelength such as UV that does not overlap with the wavelength of the infrared source 24 might be added to allow operation of the type described with respect to FIG. 2(c). In other embodiments, only two narrow band sources might be used. In the FIG. 3(a) configuration, the proximally located light sources may be on the scope or positioned separately such as on the video cart 14. Fiber couplers 30 and splitters 32 may be used inside the endoscope if needed.

FIG. 3(b) schematically shows an alternative endoscope head 12b which has the light sources 22, 24 at or around the distal end of the scope. These elements may be in a circumferential arrangement or some other arrangement.

In these embodiments, features, optical components or lens elements may be positioned to diffuse the light emitted from the light source or optical fiber, or to present an even illumination spread when the system switches between light sources.

Alternatively, or additionally, sources may be positioned to emit light from one or more devices other than the scope. For example, referring to FIG. 4, the light may emitted from the trocar 30 through which the scope 12 passes through an incision into the body. The light may be emitted from the distal part of the trocar or another location on the trocar, or from the trocar lumen. It may be emitted from LEDs or other light sources on the trocar, or from optical fibers on the trocar that carry light from a more remotely positioned light source. This may give specular light exposure from a point or series of points, or more evenly distributed light emanating from the trocar. For systems in which a plurality of instruments, including the scope, pass through a single trocar, light may emanate from locations between instrument channels of the trocar. This may be point-based, such as using an optical fiber, or it may transmit through optical elements or features to provide an even or uneven (targeted) illumination profile across the surgical field. Where the scope is used in a system employing multiple trocars, illumination may additionally or alternatively be provided from locations other than nominally inline of the endoscope. This off-axis light may come from trocars other than the one through which the endoscope passes, or from other instruments. This may be general broad-area illumination or more focused light. Light emitted from sources significantly off-axis may provide transmittance information through tissue rather than reflectance off the surface of tissue, providing tissue information or subsurface features or anatomy.

The systems and methods described here provide a scope that gives the user information beyond that which can be obtained using visible light alone. It allows the system to switch or be switched between visible light and invisible light modes (e.g. resulting in a camera display that alternates a display of an image obtained using the visible illumination source with one obtained using the source of non-visible light such as UV or IR), or a combined or hybrid image showing the primary image obtained using the visible illumination as well as enhancements to the image obtained using the non-visible light source, allowing the user to see tissue regions or structures made visible through fluorescence (e.g. tumors, blood vessels, lymph nodes, ureters) simultaneously displayed with the primary image, in each case without requiring surgical staff to switch endoscopes.

Where the system is to be used for fluorescence imaging, agents such as indocyanine green (ICG) and methylene blue could be used as known in the art to allow visualization of specific tissues or compound types such as nerves, blood etc, or to specific pathologies such as cancer. These agents may be locally administered to the tissue or administered intravenously. Other types of fluorescence, including white light fluorescence and/or autofluorescence may also be practiced using the systems and methods disclosed here.

All patents and patent applications referred to herein, included for purposes of priority, are incorporated herein by reference.

1-25. (canceled)

26. An imaging method, comprising:
positioning a multi-spectral image sensor to capture light emitted or reflected from a surgical work site within a patient;
directing light in a first wavelength range onto the work site during a first period;
using an image sensor, capturing light emitted or reflected from the work site during the first period and generating corresponding video images for display on a video display;
directing light in a second wavelength range onto the work site during a second period;
using the image sensor, capturing light emitted or reflected from the work site during the second period and generating corresponding video images for display on the video display.

27. The method of claim 26, wherein the first and second periods alternate in accordance with a predetermined duty cycle.

28. The method of claim 26, wherein the method includes receiving a user input and moving between first and second periods based on user input.

29. The method of claim 26, wherein the first and second wavelength ranges are non-overlapping ranges.

30. The method of claim 26, wherein positioning the multi-spectral imager includes inserting a scope with the multi-spectral imager thereon through a trocar disposed through an incision in the patient, and wherein at least the light in the first wavelength range is emitted from a light emitting device or an optical fiber carried on the trocar.

31. The method of claim 26 wherein positioning the multi-spectral imager includes inserting a scope with the multi-spectral imager thereon through a first incision in a patient, wherein the method includes positioning a trocar through a second incision in the patient, and wherein at least the light in the first wavelength range is emitted from a light emitting device or an optical fiber carried on the trocar.

32. The method of claim 26,
wherein the method includes
providing a medical imaging scope comprising:
the image sensor;
a first source positioned to direct light in the first wavelength range onto the surgical work site;
a second source positioned to direct light in the second wavelength range onto a surgical work site.

33. The method of claim 26, wherein the first wavelength range is in the visible range of the electromagnetic spectrum, and the second wavelength range is in the infrared range or ultraviolet range of the electromagnetic spectrum.

34. The method of claim 33, wherein the second range is in the near infrared range.

35. The method of claim 33, wherein light from the first source is reflected off of objects in the work site and captured by the image sensor, and light from the second source is absorbed by material in the worksite and emitted in the form of fluorescence, said emitted light captured by the image sensor.

36. The method of claim 32, wherein the first source is a broad spectrum source and the second source is a narrow spectrum source.

37. The method of claim 32, wherein at least one of the first and second sources is a tunable source.

38. The method of claim 26, wherein the image sensor is provided to include a filter array having a plurality of filters, each positioned over a particular pixel of the image sensor, each filter selected to allow transmission of light from a particular range of the electromagnetic spectrum to pass to the underlying pixel.

39. The method of claim 38, wherein the filter array includes first filters transmissive to red light, second filters transmissive to blue light, third filters transmissive to green light, and fourth filters transmissive to infrared light.

40. The method of claim 39, wherein the filter array includes a first part and a second part, wherein the arrangement of the first, second, third and fourth filters is different in the first part than in the second part.

41. The method of claim 26, further including scope of claim 3, further including directing light in a third wavelength range onto a work site and, using the image sensor to capture light emitted or reflected from the work site during illumination of the first source, during illumination of the second source, and during illumination of the third source.

42. The method of claim 41, wherein light from the first source is reflected off of objects in the work site and captured by the image sensor, light from the second source is absorbed by first fluorescent material in the worksite and emitted in the form of first fluorescence, light from the third source is absorbed by second fluorescent material in the worksite and emitted in the form of second fluorescence, said emitted light captured by the image sensor.

43. The method of claim 26, wherein the first wavelength range is in the infrared range of the electromagnetic spectrum, and the second wavelength range is in the ultraviolet range of the electromagnetic spectrum.

44. The method of claim 45, further including directing light in a third wavelength range onto a work site and, using the image sensor to capture light emitted or reflected from the work site during illumination of the first source, during illumination of the second source, and during illumination of the third source, wherein the third wavelength range is in the visible range of the electromagnetic spectrum.

45. The method of claim 32, wherein the first and second sources are a single tunable light source
